(19) United States
(12) Patent Application Publication
(10) Pub. No.: US 2010/0160653 A1
(43) Pub. Date: Jun. 24, 2010

(54) DOCETAXEL POLYMORPHS AND PROCESSES

(75) Inventors: Raghavendracharyulu Venkata Palle, Hyderabad (IN); Sekhar Munaswamy Nariyam, Hyderabad (IN); Selvakumar Balaraman, Theni (IN); Neelakandan Kaliaparumal, Cuddalore (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad, 500 016, Andra Pradesh (IN); DR. REDDY'S LABORATORIES, INC., Bridgewater, NJ (US)

(21) Appl. No.: 12/293,678

(22) PCT Filed: Mar. 20, 2007
(86) PCT No.: PCT/US07/64375

§ 371 Date: Sep. 19, 2008

Related U.S. Application Data
(60) Provisional application No. 60/807,361, filed on Jul. 14, 2006.
(60) Provisional application No. 60/824,950, filed on Sep. 8, 2006.
(60) Provisional application No. 60/893,150, filed on Mar. 6, 2007.

(30) Foreign Application Priority Data
Mar. 21, 2006 (IN) 502/CHE/2006
Jul. 25, 2006 (IN) 1291/CHE/2006
Dec. 5, 2007 (IN) 2252/CHE/2006

(51) Int. Cl. C07D 305/14

(52) U.S. Cl. 549/510

(57) ABSTRACT

The present invention provides crystalline polymorphs of docetaxel and processes for preparing them, a method for preparing amorphous docetaxel, and a process for preparing docetaxel.

### INTRODUCTION TO THE INVENTION

The present invention relates to polymorphs of docetaxel, processes for preparing them and methods of using them to make other docetaxel polymorphs. The present invention also provides a process for the preparation of docetaxel.

Docetaxel is the adopted name for a drug compound having a chemical name (2R,3S)-N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate and having the structural Formula I.

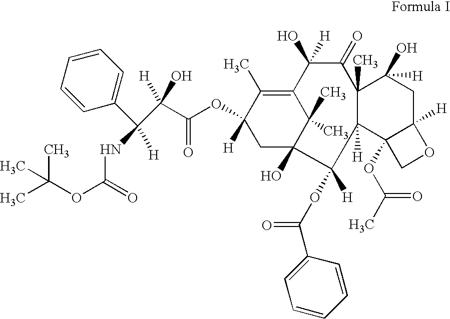

Docetaxel is an antineoplastic agent belonging to the taxoid family and is available in the market under the brand name TAXOTERE® in the form of a sterile, non-pyrogenic injection in single-dose vials containing 20 mg (0.5 mL) or 80 mg (2 mL) of the drug.

A number of taxol analogs have been described by F. Guéritte-Voegelein et al., “Relationships between the Structure of Taxol Analogues and Their Antimitotic Activity,” Journal of Medicinal Chemistry, Vol. 34, pages 992-998, 1991.

U.S. Pat. No. 4,814,470 discloses docetaxel, its stereo isomeric forms, pharmaceutical compositions containing docetaxel and their use in the treatment of acute leukaemias and solid tumours.

U.S. Pat. No. 6,197,980 discloses docetaxel trihydrate and a process for its preparation.

Processes for the preparation of docetaxel trihydrate have been described in U.S. Pat. No. 6,022,985 and U. S. Patent Application Publication No. 2006/0217436.

U.S. Patent Application Publication No. 2005/0065138 discloses an acetone solvate of dimethoxy docetaxel and a process for its preparation.

International Application Publication No. WO 2005/061474 discloses a process for the preparation of anhydrous, amorphous and tri-hydrate forms of docetaxel.

U.S. Pat. No. 6,838,569 discloses a process for the purification of docetaxel trihydrate, comprising the dissolution of docetaxel in acetonitrile followed by precipitation with purified water.

U.S. Pat. No. 6,002,025 discloses a process for the purification of taxanes by using column chromatography containing phenyl alkyl resin.

U.S. Pat. No. 5,476,954 discloses a process for the preparation of docetaxel and its derivatives.

U.S. Pat. No. 5,532,388 discloses a process for the preparation of taxoide.

Regulatory authorities require that efforts be made to identify all polymorphic forms, e.g., crystalline, amorphous, solvated, etc., of new drug substances, since polymorphic forms can vary in their chemical and physical properties. This variation frequently results in bioavailability, stability, and other differences between production lots of formulated pharmaceutical products.

However, the existence, and possible numbers, of polymorphic forms for a given compound cannot be predicted. In addition, there are no “standard” procedures that can be used to prepare polymorphic forms of a substance.

Therefore, there is a need for preparing new solid forms of a drug substance and processes for preparation thereof.

According to the present invention there is provided a convenient process for the preparation of docetaxel and its polymorphs with desired purity and yield by using better preparation techniques, which are simple, ecofriendly, cost-effective, robust and well suited for use on an industrial scale.

### SUMMARY OF THE INVENTION

The present invention relates to polymorphs of docetaxel and processes for preparing them. The present invention also provides a process for the preparation of docetaxel.

In an aspect, the present invention relates to docetaxel polymorphs and process thereof.

In an embodiment, the present invention provides crystalline polymorphs of docetaxel and process for preparing them. These polymorphs are hereinafter referred to as Form I, Form II, Form III, Form IV, Form V, Form VI, Form VII, Form VIII, and Form IX.

Another embodiment of the present invention relates to a process for preparing an amorphous form of docetaxel comprising precipitating amorphous docetaxel from a solution of docetaxel in tetrahydrofuran (THF) by combining with an antisolvent such as a hydrocarbon and recovering a precipitated amorphous solid.

In another aspect, the present invention provides a process for the preparation of docetaxel comprising:

a) reacting the compound 4-acetoxy-2α-benzoyloxy-5β,20-epoxy-1-hydroxy-9-oxo-7β,10β-bis(2,2,2-trichloroethoxycarbonyloxy)-tax-11-en-13-α-yl(4S,5R)-3-t-(butoxycarbonyl)-2,2-dimethyl-4-phenyl-5-oxazolidinecarboxylate (DCT-II) of Formula V with zinc and acetic acid to form the compound 4-acetoxy-2α-benzoyloxy-5β,20-epoxy-1β,7β,10β-trihydroxy-9-oxo-tax-11-en-13-α-yl-(4S,5R)-3-t-(butoxycarbonyl)-2,2-dimethyl-4-phenyl-5-oxazolidinecarboxylate (DCT-III) of Formula VI;

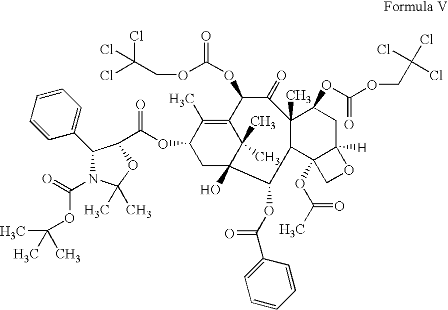

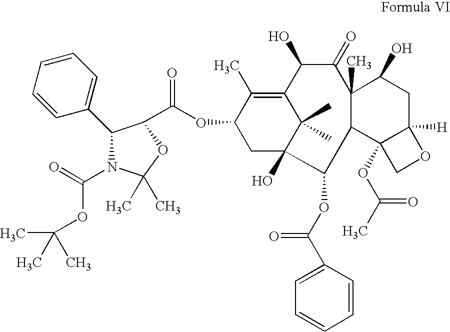

b) reacting the DCT-III compound of Formula VI with an acid to form the compound 4-acetoxy-2α-benzoyloxy-5β,20-epoxy-1β,7β,10β-trihydroxy-9-oxo-tax-11-en-13-α-yl(2R,3S)-3-amino-2-hydroxy-3-phenyl-propionate (DCT-IV) of Formula VIA; and

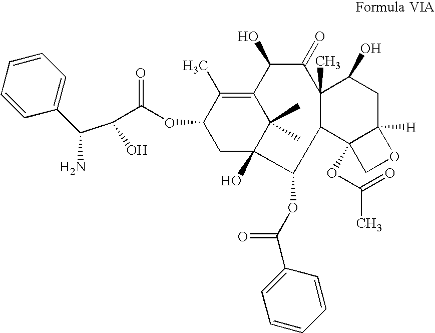

c) reacting the DCT-IV compound of Formula VIA with di-t-butyl dicarbonate to form docetaxel of Formula I.

Further, an aspect of the present invention provides a pharmaceutical composition comprising one or more of crystalline Form I, Form II, Form III, Form IV, Form V, Form VI, Form VII, Form VIII, and Form IX, and an amorphous form, of docetaxel along with one or more pharmaceutically acceptable excipients.

The docetaxel polymorphs of present invention are stable and are well suitable for pharmaceutical formulations, which are useful in the treatment of disease, including, but are not limited to, neoplastic tumors.

In an embodiment, the invention provides a process for preparing docetaxel, comprising reacting a compound having a formula:

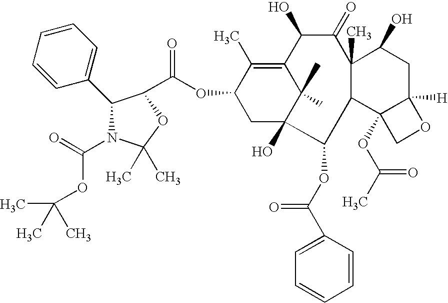

with an acid, to form a compound having a formula:

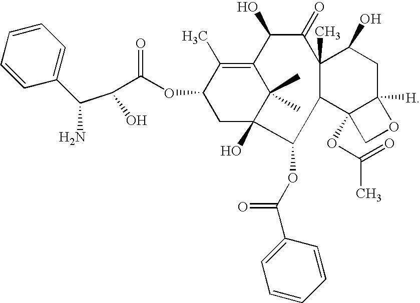

In another embodiment, the invention provides a process for preparing crystalline Form I of docetaxel, comprising combining a solution of docetaxel in a ketone with an anti-solvent.

A further embodiment of the invention provides process for preparing crystalline Form II of docetaxel, comprising combining a solution of docetaxel in acetonitrile with water.

In a still further embodiment, the invention provides a process for preparing crystalline Form III of docetaxel, comprising slurrying solid docetaxel in isopropyl alcohol.

A yet further embodiment of the invention provides a process for preparing crystalline Form IV of docetaxel, comprising combining a solution of docetaxel in N,N-dimethylformamide with water.

In an additional embodiment, the invention provides a process for preparing crystalline Form V of docetaxel, comprising combining a solution of docetaxel in tetrahydrofuran with toluene.

An embodiment of the invention provides a process for preparing a crystalline polymorphic form of docetaxel, comprising:

a) providing a solution of docetaxel in an organic solvent;

b) removing solvent from the solution of a) for formation of crystals; and

c) recovering a solid crystalline polymorph of docetaxel.

Other embodiments of the invention provide: crystalline Form I of docetaxel; crystalline Form II of docetaxel; crystalline Form III of docetaxel; crystalline Form IV of docetaxel; crystalline Form V of docetaxel; crystalline Form VI of docetaxel; crystalline Form VII of docetaxel; crystalline Form VIII of docetaxel; and crystalline Form IX of docetaxel.

In a still further embodiment, the invention provides a process for preparing solid amorphous docetaxel, comprising combining a solution of docetaxel in tetrahydrofuran with an anti-solvent.

In a yet further embodiment, the invention provides a process for preparing solid amorphous docetaxel, comprising removing solvent from a solution of docetaxel in an alcohol.

### BRIEF DESCRIPTION OF THE DRAWINGS

FIG. 1 is a schematic representation of a process for preparing docetaxel.

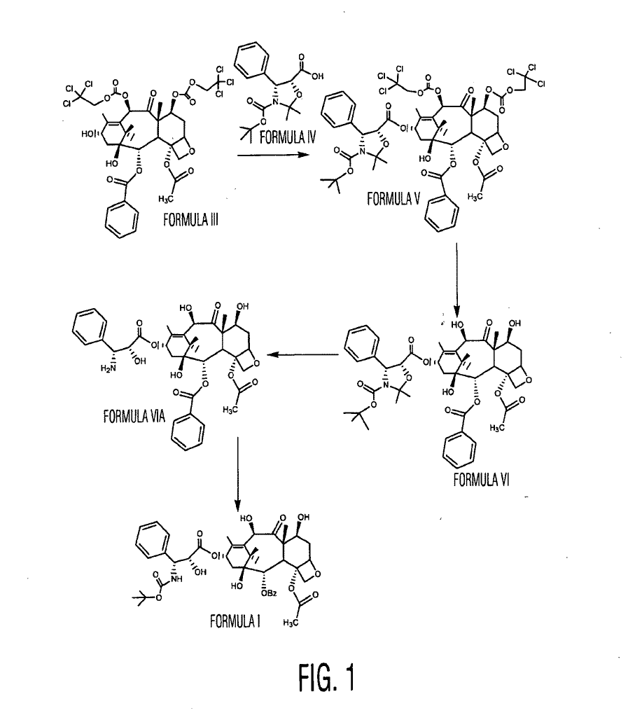

FIG. 2 is an X-ray powder diffraction (XRPD) pattern of docetaxel Form I prepared according to Example 18.

FIG. 3 is a differential scanning calorimetry (“DSC”) curve of docetaxel Form I prepared according to Example 18.

FIG. 4 is a thermogravimetric analysis (TGA) curve of docetaxel Form I prepared according to Example 18.

FIG. 5 is an XRPD pattern of docetaxel Form II prepared according to Example 2.

FIG. 6 is a DSC curve of docetaxel Form II prepared according to Example 2.

FIG. 7 is a TGA curve of docetaxel Form II prepared according to Example 2.

FIG. 8 is an XRPD pattern of docetaxel Form III prepared according to Example 3.

FIG. 9 is a DSC curve of docetaxel Form III prepared according to Example 3.

FIG. 10 is a TGA curve of docetaxel Form III prepared according to Example 3.

FIG. 11 is an XRPD pattern of docetaxel Form IV prepared according to Example 4.

FIG. 12 is a DSC curve of docetaxel Form IV prepared according to Example 4.

FIG. 13 is a TGA curve of docetaxel Form IV prepared according to Example 4.

FIG. 14 is a XRPD pattern of docetaxel Form V prepared according to Example 5.

FIG. 15 is a DSC curve of docetaxel Form V prepared according to Example 5.

FIG. 16 is a TGA curve of docetaxel Form V prepared according to Example 5.

FIG. 17 is a XRPD pattern of docetaxel Form VI prepared according to Example 8.

FIG. 18 is a DSC curve of docetaxel Form VI prepared according to Example 8.

FIG. 19 is a TGA curve of docetaxel Form VI prepared according to Example 8.

FIG. 20 is an XRPD pattern of docetaxel Form VII prepared according to Example 9.

FIG. 21 is a DSC curve of docetaxel Form VII prepared according to Example 9.

FIG. 22 is a TGA curve of docetaxel Form VII prepared according to Example 9.

FIG. 23 is an XRPD pattern of docetaxel Form VIII prepared according to Example 10.

FIG. 24 is a DSC curve of docetaxel Form VIII prepared according to Example 10.

FIG. 25 is a TGA curve of docetaxel Form VIII prepared according to Example 10.

FIG. 26 is an XRPD pattern of docetaxel Form IX prepared according to Example 11.

FIG. 27 is a DSC curve of docetaxel Form IX prepared according to Example 11.

FIG. 28 is a TGA curve of docetaxel Form IX prepared according to Example 11.

FIG. 29 is an ORTEP pattern of docetaxel Form VIII prepared according to Example 10.

FIG. 30 is an ORTEP pattern of docetaxel Form IX prepared according to Example 11.

FIG. 31 is an XRPD pattern of amorphous docetaxel prepared according to Example 6.

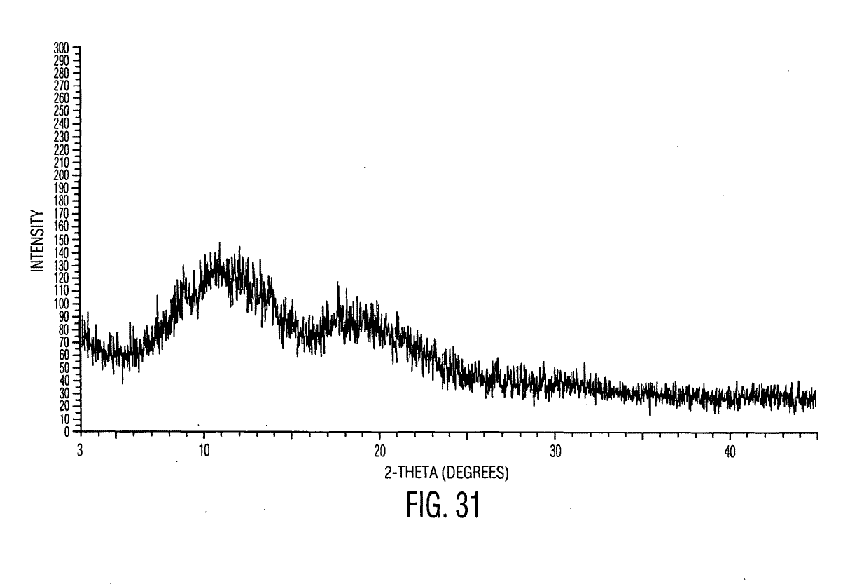

### DETAILED DESCRIPTION OF THE INVENTION

The present invention relates to polymorphic forms of docetaxel and processes for preparing them. The present invention also provides a process for the preparation of docetaxel.

In one embodiment, the present invention provides crystalline polymorphs of docetaxel and process for preparing them. These polymorphs are hereinafter referred to as Form I, Form II, Form III, Form IV, Form V, Form VI, Form VII, Form VIII, and Form IX.

The crystalline polymorphs obtained by the process of present invention, unless stated otherwise, are characterized by their X-ray powder diffraction (“XRPD”) patterns, differential scanning calorimetry (“DSC”) curves, and termogravimetric analysis (TGA) curves.

All XRPD data reported herein were obtained using Cu Kα radiation, having the wavelength 1.541 A and were obtained using a Bruker AXS D8 Advance Powder X-ray Diffractometer.

Differential scanning calorimetric analysis was carried out in a DSC Q1000 instrument from TA Instruments with a ramp of 5° C./minute with a modulation time of 60 seconds and a modulation temperature of ±1° C. The starting temperature was 0° C. and ending temperature was 200° C.

TGA analysis was carried out in a TGAQ500V64 Build 193 instrument with a ramp 10° C./minute up to 250° C.

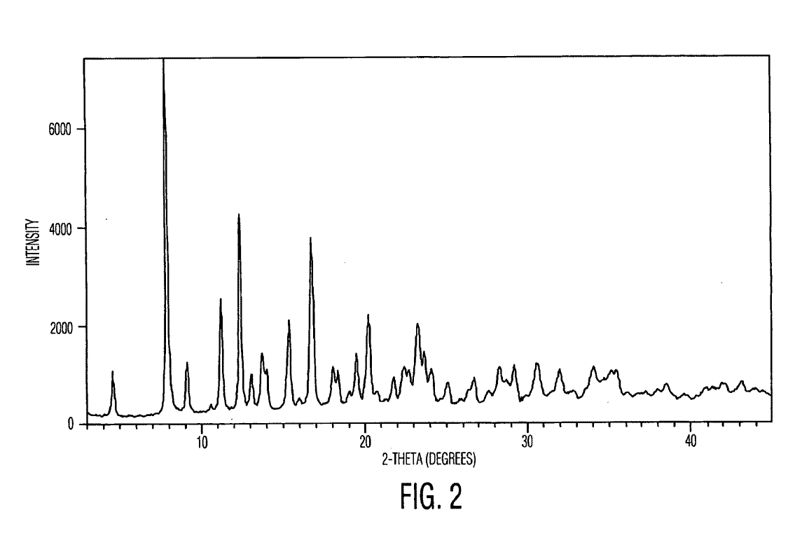

Docetaxel crystalline Form I of the present invention is characterized by its XRPD pattern, which is substantially in accordance with FIG. 2. Docetaxel crystalline Form I of the present invention is characterized by its XRPD pattern having characteristic peaks at diffraction angles 2-theta of about 8.0, 11.3, 12.5, 13.8, 15.4, 16.9, 20.3, and 23.3, ±0.2 degrees.

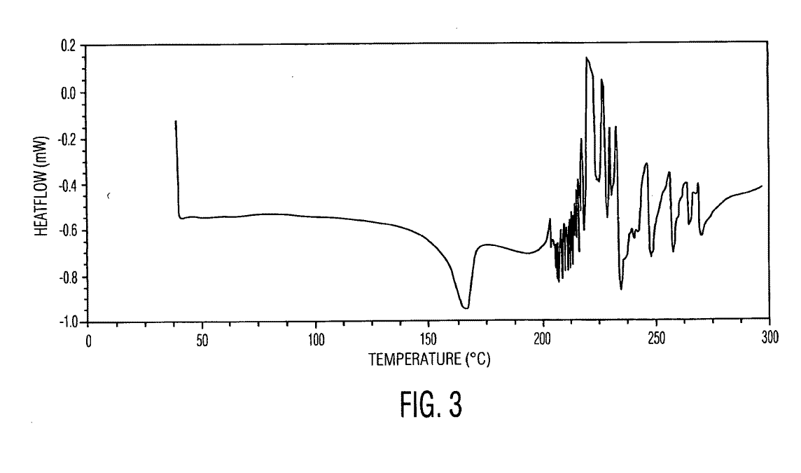

Docetaxel crystalline Form I of the present invention is further characterized by its DSC curve substantially in accordance with FIG. 3, having an endothermic peak at about 165.05° C.

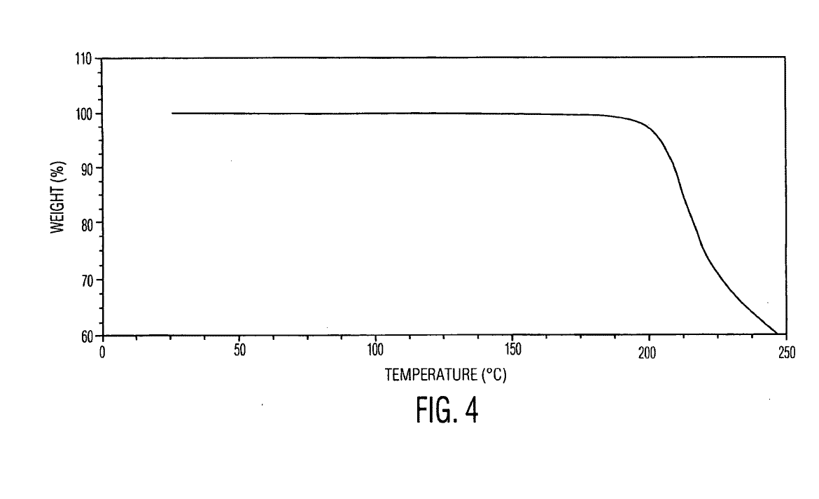

Docetaxel crystalline Form I of the present invention is further characterized by its TGA curve substantially in accordance with FIG. 4 corresponding to a weight loss of about 0.2% w/w.

The present invention provides a process for the preparation of docetaxel crystalline Form I comprising combining a solution of docetaxel in a ketone with an anti-solvent.

The solution of docetaxel is obtained by the dissolution of docetaxel in a suitable ketone such as acetone, methyl isobutyl ketone, methyl ethyl ketone, and the like.

The concentration of docetaxel in the solution is not critical as long as sufficient solvent is employed to ensure total dissolution. The amount of solvent employed is usually kept as small as possible, to avoid excessive product losses during crystallization and isolation.

The quantity of solvent used for preparing the solution depends on the nature of the solvent and the temperature adopted for preparing the solution. The concentration of docetaxel in the solution may generally range from about 0.01 to about 0.15 g/ml in the solvent.

Suitable temperatures for preparation of the solution can range from about 20 to 120° C., or to about 25 to about 35° C., depending on the solvent used. Any other temperature is also acceptable as long as the stability of docetaxel is not compromised.

Docetaxel is crystallized from the solution by combining with an anti-solvent. Suitable anti-solvents include but are not limited to: ethers such as diethyl ether, diisopropyl ether, 1,4-dioxane, dimethoxyethane, methyl tertiary-butyl ether and the like; hydrocarbons such as n-pentane, n-hexane, n-heptane, cyclohexane, benzene, toluene, and the like; low boiling hydrocarbon mixtures such as petroleum ether and the like; and combinations thereof.

The ratio between the solvent in solution and anti-solvent is from about 1:1 to about 1:10 or about 1:3 by volume.

The obtained Form I is optionally slurried in a suitable solvent to reduce the organic volatile impurities content. Suitable solvents used for forming a slurry include n-heptane, n-hexane, cyclohexane and the like. Suitable temperature can range from 20 to about 40° C., or from about 25 to about 35° C. The slurry can be maintained for periods of about 20 minutes to about 24 hours, or longer.

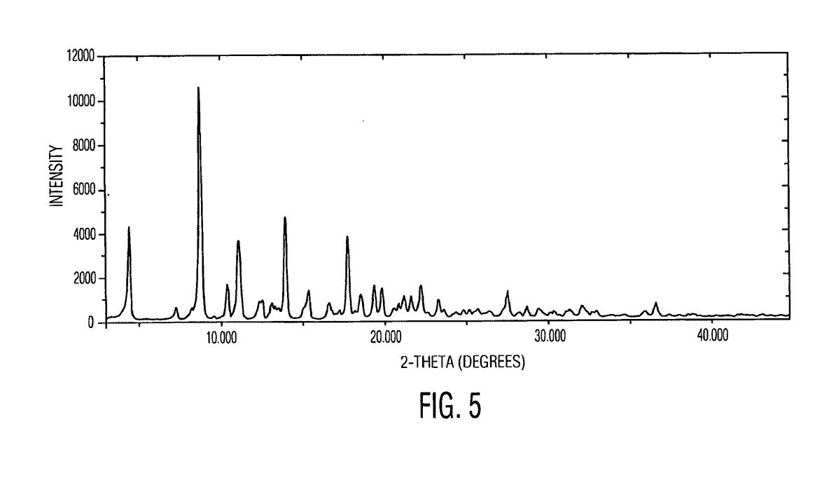

Docetaxel crystalline Form II of the present invention is characterized by its XRPD pattern, which is substantially in accordance with FIG. 5. Docetaxel crystalline Form II of the present invention is characterized by its XRPD pattern having characteristic peaks at diffraction angles 2-theta of about 4.4, 7.2, 8.8, 10.4, 11.1, 14, 17.8, and 19.4, ±0.2 degrees.

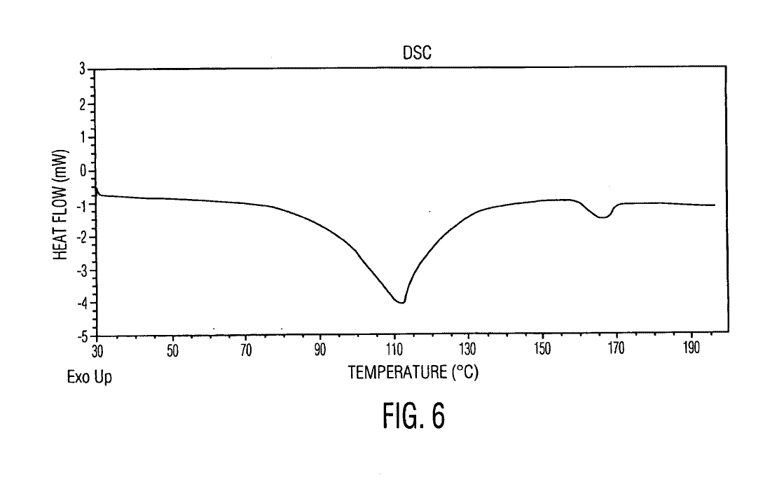

Docetaxel crystalline Form II is further characterized by its DSC curve, which is substantially in accordance with FIG. 6, having endothermic peaks at about 112 and 166° C.

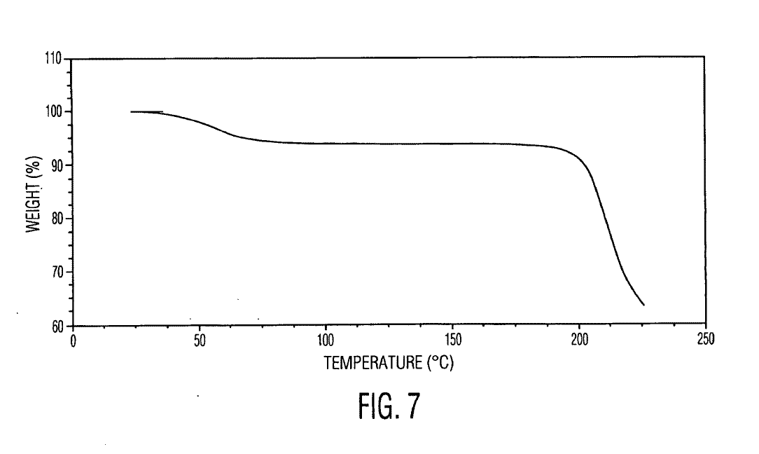

Docetaxel crystalline Form II of the present invention has a TGA curve substantially in accordance with FIG. 7 corresponding to a weight loss of about 6% w/w. It has water content of about 7% w/w by the KF method

The present invention provides a process for the preparation of docetaxel crystalline Form II comprising combining a solution of docetaxel in acetonitrile with water at suitable temperatures.

The solution of docetaxel is obtained by dissolving docetaxel in acetonitrile. The temperatures for preparation of the solution can range from about 20 to 120° C., or to about 40 to about 45° C. The concentration of docetaxel in the solution may generally range from about 0.05 to about 0.5 g/ml or 0.1 g/ml.

Docetaxel is crystallized from the solution by combining with an anti solvent such as water. The ratio between the acetonitrile in the solution and anti solvent is from about 1:1 to about 1:10, or from about 1:4 to about 1:5, by volume.

Suitable temperatures for the crystallization of docetaxel Form II are from about 25 to about 70° C., or about 40 to about 45° C.

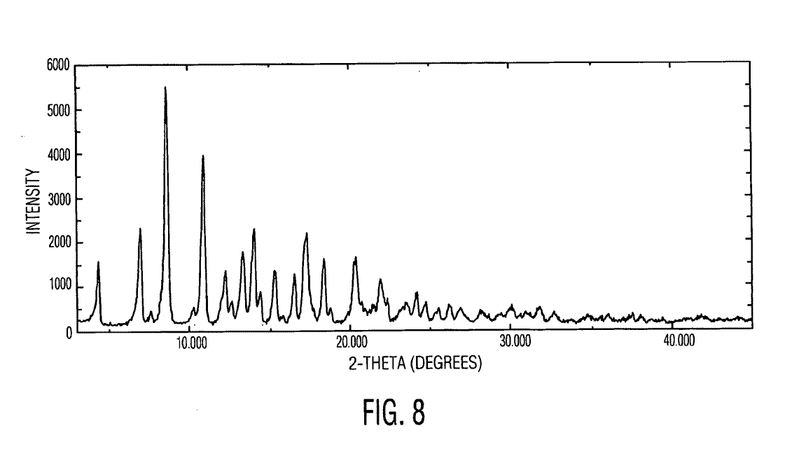

Docetaxel crystalline Form III of the present invention is characterized by its XRPD pattern, which is substantially in accordance with FIG. 8. Docetaxel crystalline Form III of the present invention is characterized by its XRPD pattern having characteristic peaks at diffraction angles 2-theta of about 4.3, 7.0, 8.7, 11, 12.3, 13.3, 14, 17.2, 17.3, 18.4, and 20.4, ±0.2 degrees.

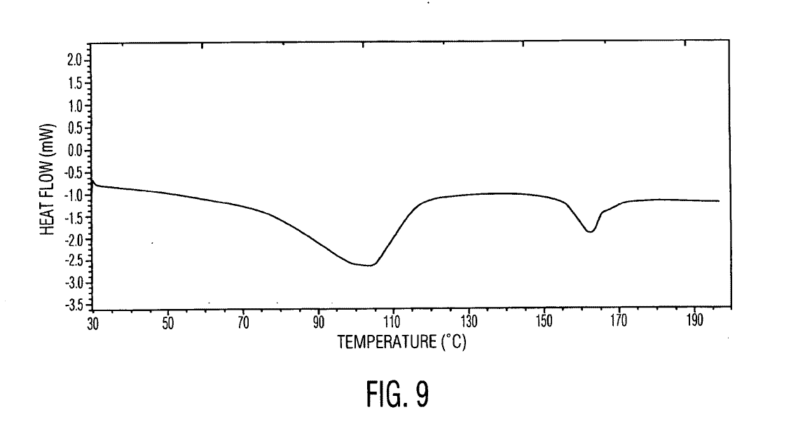

Docetaxel crystalline Form III is further characterized by its DSC curve, which is substantially in accordance with FIG. 9, having endothermic peaks at about 104 and 162° C.

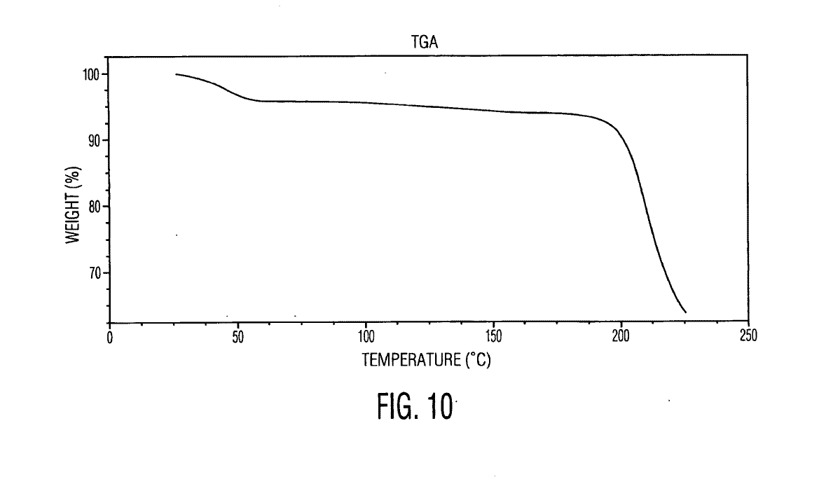

Docetaxel crystalline Form Ill of the present invention has a characteristic TGA curve substantially in accordance with FIG. 10 corresponding to a weight loss of about 6% w/w. It has water content about 6% w/w by the KF method.

The present invention provides a process for the preparation of docetaxel crystalline Form III comprising slurrying the docetaxel in isopropyl alcohol for a period of about 30 minutes to about 5 hours, or about 1 hour, or longer.

The quantity of isopropyl alcohol may generally range from about 1 L to about 5 L, or about 2 L, per kilogram of docetaxel in solution.

Suitable temperature for the slurrying range from about 20 to about 60° C. or from about 25 to about 35° C.

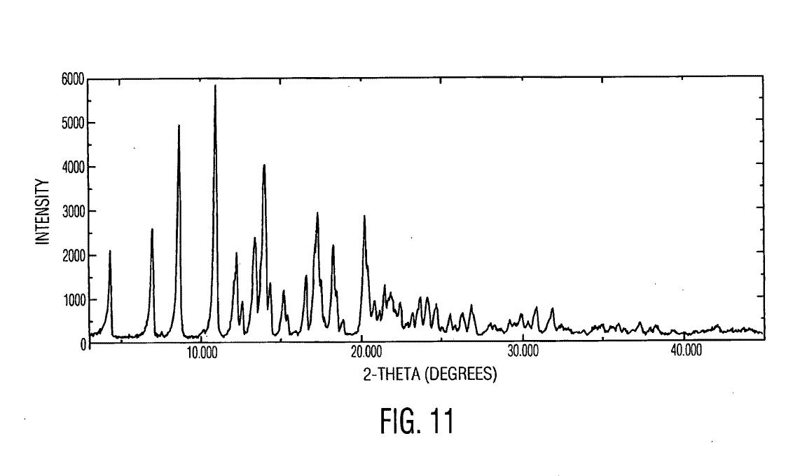

Docetaxel crystalline Form IV of the present invention is characterized by its XRPD pattern, which is substantially in accordance with FIG. 11. Docetaxel crystalline Form IV of the present invention is characterized by its XRPD pattern having characteristic peaks at diffraction angles 2-theta about 4.3, 7.0, 8.7, 10.9, 12.2, 13.4, 14, 17.1, 17.2, 18.2, 20.2, and 20.4, ±0.2 degrees.

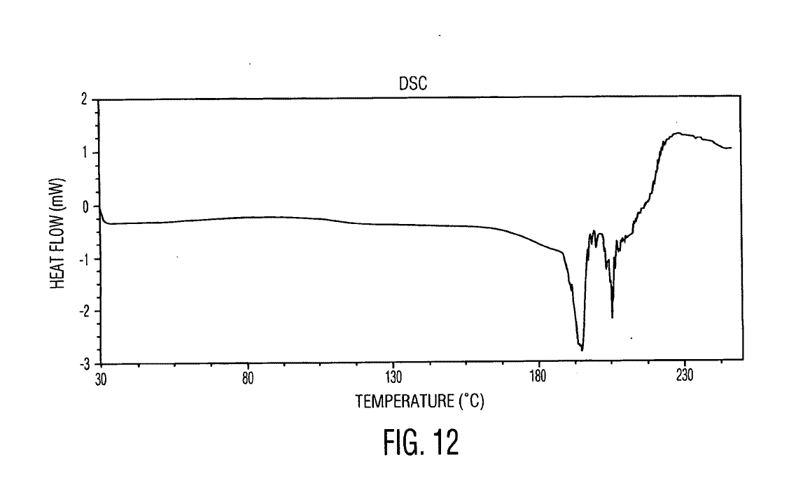

Docetaxel crystalline Form IV is further characterized by its DSC curve, which is substantially in accordance with FIG. 12, having endothermic peaks at about 114 and 195° C.

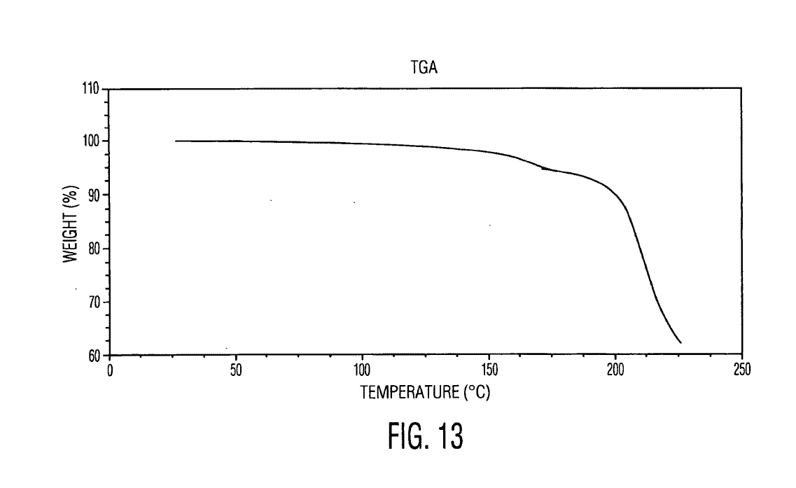

Docetaxel crystalline Form IV of the present invention has a characteristic TGA curve substantially in accordance with FIG. 13 corresponding to a weight loss of about 6% w/w. It has a water content about 1 w/w by the KF method.

The present invention provides a process for the preparation of docetaxel crystalline Form IV comprising combining a solution of docetaxel in N,N-dimethylformamide (“DMF”) with water. The solution of docetaxel can be prepared by the dissolution of docetaxel in

DMF. The amount of docetaxel dissolved depends on the solvent volume and on temperature. The concentration of docetaxel in the solution can range from about 0.1 to about 1 g/ml, or about 0.5 g/ml.

Docetaxel crystalline Form IV is precipitated by combination of water with the solution of docetaxel in DMF.

The ratio between the DMF and the water can be range from about 1:1 to about 1:15 or to about 1:10, by volume.

Suitable temperatures for the formation of docetaxel Form IV are from about 20 to about 60° C., or from about 25 to about 35° C.

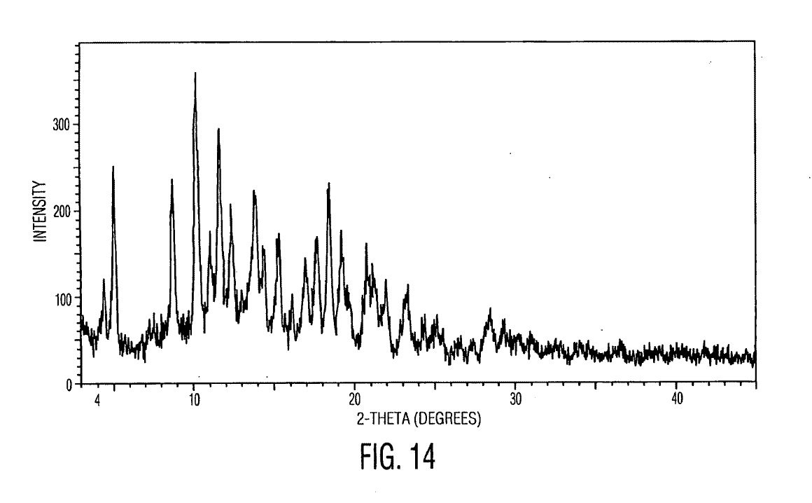

Docetaxel crystalline Form V of the present invention is characterized by its XRPD pattern, which is substantially in accordance with FIG. 14. Docetaxel crystalline Form V of the present invention is characterized by its XRPD pattern having characteristic peaks at diffraction angles 2-theta of about 4.4, 5.1, 8.8, 10.3, 11.1, 11.7, 12.4, 13.9, 14.4, 15.3, 17.0, 17.7, 18.5, 19.3, 20.8, 21.2, and 22, ±0.2 degrees.

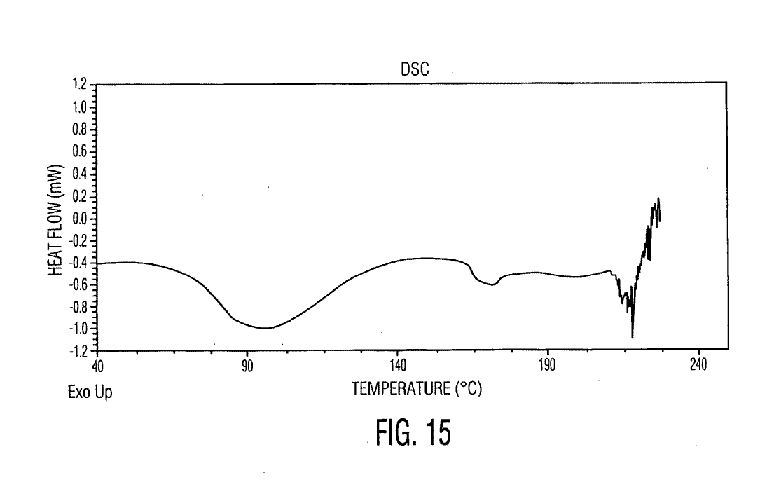

Docetaxel crystalline Form V is further characterized by its DSC curve, which is substantially in accordance with FIG. 15, having endothermic peaks at about 96 and 167° C.

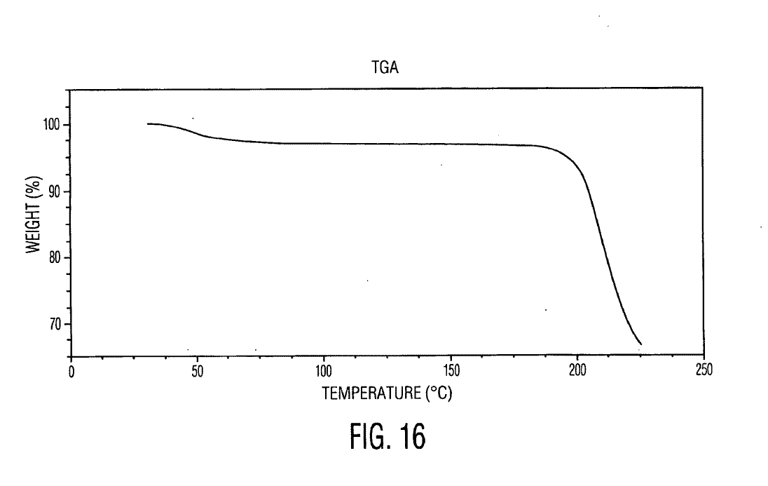

Docetaxel crystalline Form V of the present invention has a characteristic TGA curve substantially in accordance with FIG. 16 corresponding to a weight loss of about 3% w/w. It has water content about 4% w/w by the KF method.

The present invention provides a process for the preparation of docetaxel crystalline Form V comprising combining a solution of docetaxel in tetrahydrofuran with toluene.

The solution of docetaxel can be prepared by the dissolution of docetaxel in tetrahydrofuran. The amount of docetaxel dissolved depends on the volume of solvent and on temperature. The concentration of docetaxel in the solution can range from about 0.1 to about 0.5 g/ml, or about 0.25 g/ml.

Precipitation of docetaxel crystalline Form V can be carried out by combining a docetaxel solution with toluene.

The ratio between THF and toluene can range from about 1:1 to about 1:35, or about 1:20 to about 1:30, by volume.

Suitable temperatures for the formation of docetaxel crystalline Form V are from about 20 to about 60° C., or from about 25 to about 35° C.

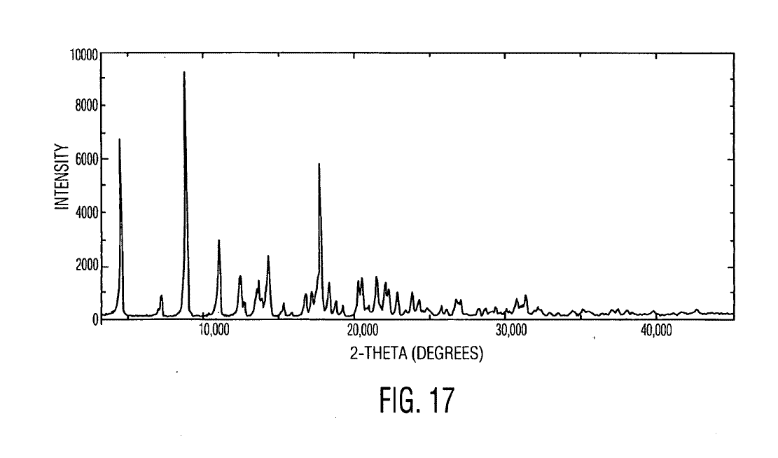

Docetaxel crystalline Form VI of the present invention is characterized by its XRPD pattern, which is substantially in accordance with FIG. 17. Docetaxel crystalline Form VI of the present invention is characterized by its XRPD pattern having characteristic peaks at diffraction angles 2-theta about 4.3, 8.7, 10.8, 12.2, 14.1, 17.4, 17.6, 20.3, 21.3, and 43.7, ±0.2 degrees.

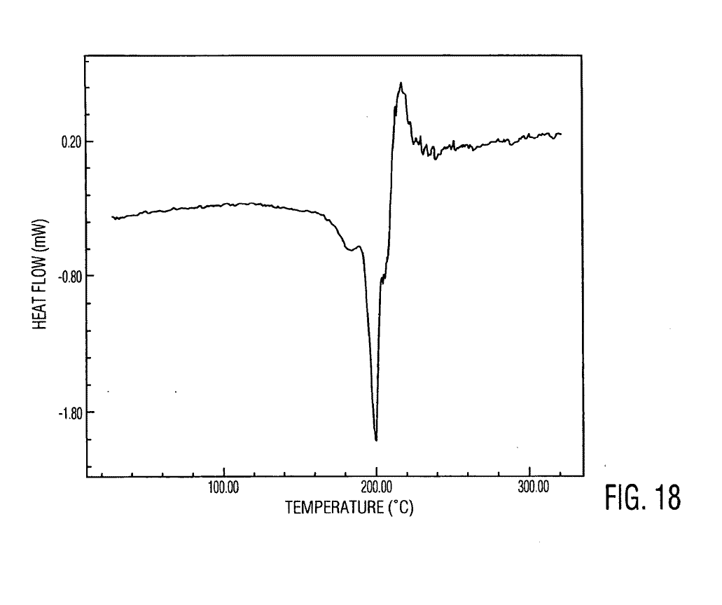

Docetaxel crystalline Form VI is further characterized by its DSC curve, which is substantially in accordance with FIG. 18, having an endothermic peak at about 200° C.

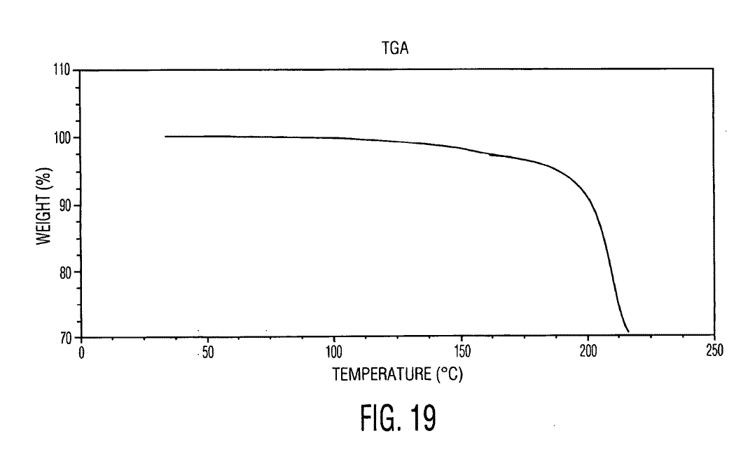

Docetaxel crystalline Form VI of the present invention has a characteristic TGA, which is substantially in accordance with FIG. 19 corresponding to a weight loss of about 4% w/w.

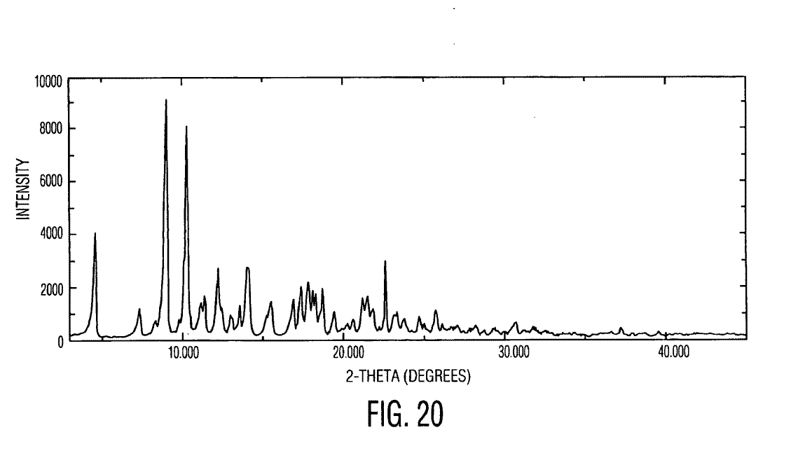

Docetaxel crystalline Form VII of the present invention is characterized by its XRPD pattern, which is substantially in accordance with FIG. 20. Docetaxel crystalline Form VII of the present invention is characterized by its XRPD pattern having characteristic peaks at diffraction angles 2-theta of about 4.6, 9.1, 10.3, 12.2, 14.1, 17.4, 17.8, 18.1, 18.7, and 22.6, ±0.2 degrees.

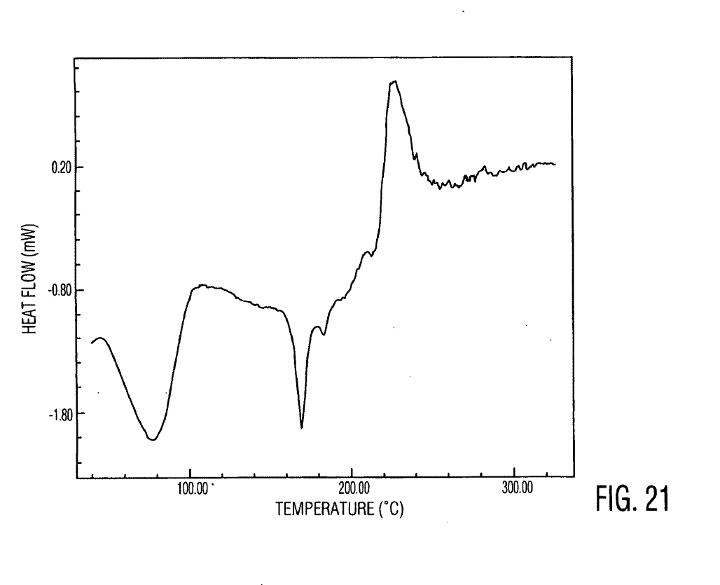

Docetaxel crystalline Form VII is further characterized by its DSC curve, which is substantially in accordance with FIG. 21, having an endothermic peak at about 183° C.

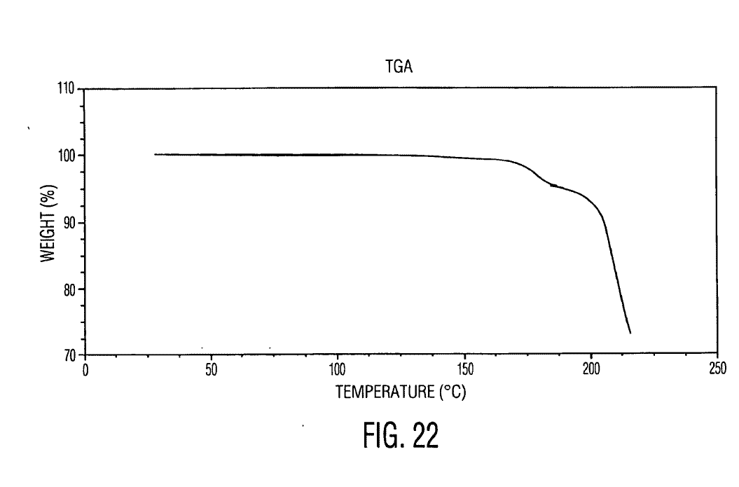

Docetaxel crystalline Form VII of the present invention has a characteristic TGA curve that is substantially in accordance with FIG. 22 corresponding to a weight loss of about 4% w/w.

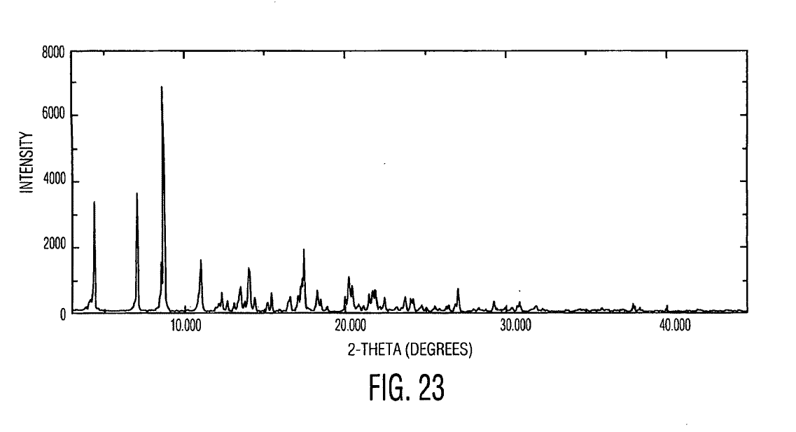

Docetaxel crystalline Form VIII is characterized by its XRPD pattern, which is substantially in accordance with FIG. 23. Docetaxel crystalline Form VIII is characterized by its XRPD pattern having characteristic peaks at diffraction angles 2-theta about 4.4, 7.0, 8.7, 11.0, 14.0, 17.5, and 20.3, ±0.2 degrees.

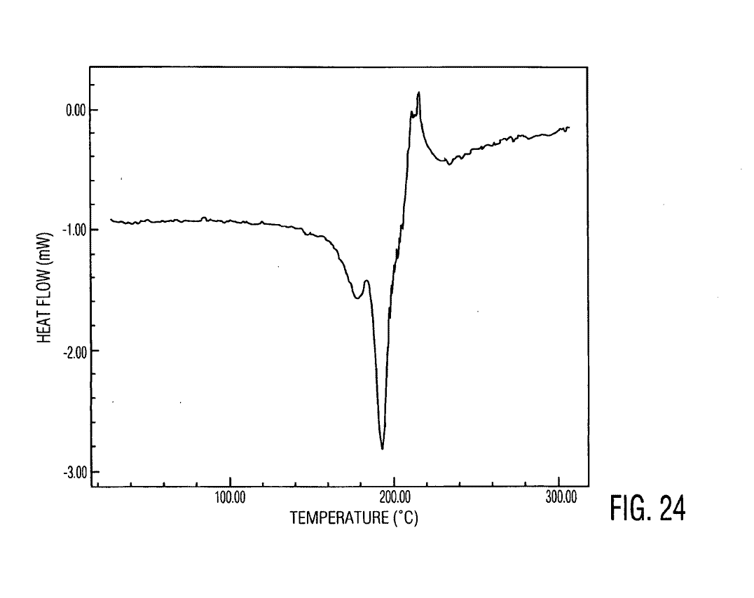

Docetaxel crystalline Form VIII is further characterized by its DSC curve, which is substantially in accordance with FIG. 24, having an endothermic peak at about 193° C.

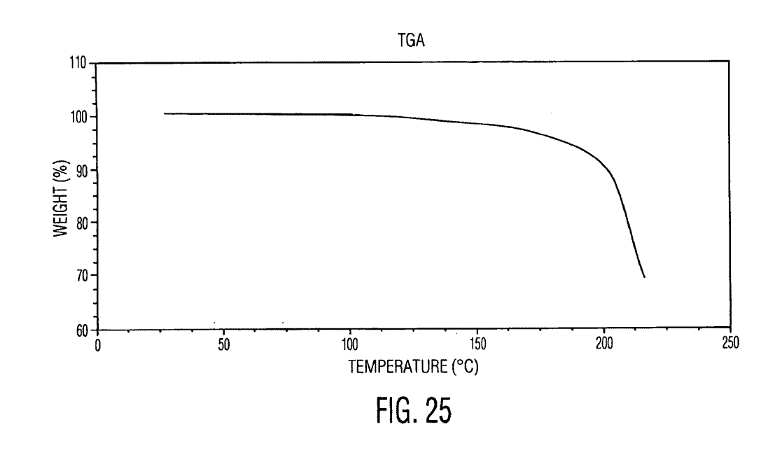

Docetaxel crystalline Form VIII of the present invention has a characteristic TGA curve, which is substantially in accordance with FIG. 25 corresponding to a weight loss of about 1% w/w.

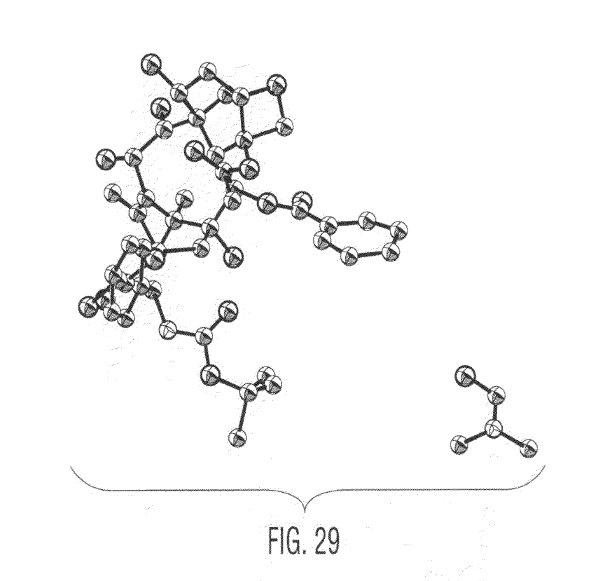

Docetaxel crystalline Form VIII is further characterized by its single crystal X-ray diffraction data (“ORTEP”) substantially in accordance with FIG. 29 and has the following characteristics

Unit Cell Parameters

| | |
|---|---|
| a | 12.804(5) |
| b | 8.743(3) |
| c | 20.587(7) |
| alpha | 90 |
| beta | 100.126(4) |
| gamma | 90 |
| Volume | 2268(1) |

Space group information

| | |
|---|---|
| Symbol | P21 |
| Number | 4 |
| Centricity | Acentric |
| Z value | 2 |
| Formula weight | 880.98 |
| calculated density | 1.290 |
| mu (cm⁻¹) | 0.962 |
| crystal system | monoclinic |
| Laue group | 2/m |
| lattice type | P |

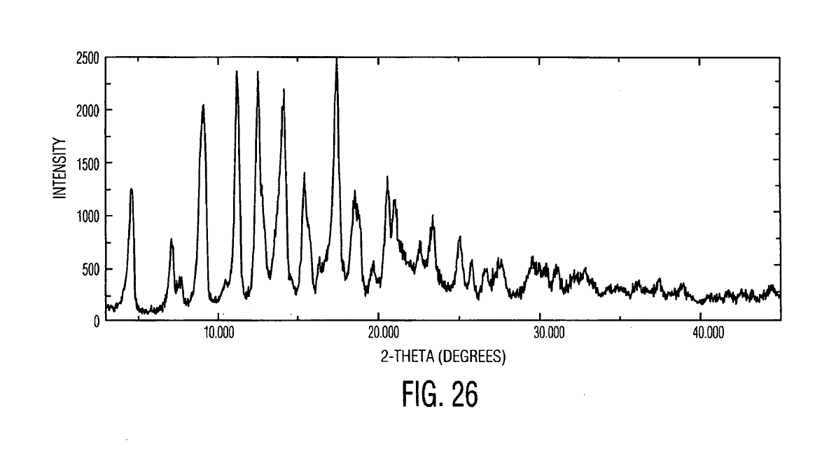

Docetaxel crystalline Form IX is characterized by its XRPD pattern substantially in accordance with FIG. 26. Docetaxel crystalline Form IX is characterized by its XRPD pattern having characteristic peaks at diffraction angles 2-theta of about 4.6, 9.2, 11.3, 12.5, 14.2, 15.4, 17.1, 17.5, 18.4, 18.6, 18.8, 20.6, and 21.0, ±0.2 degrees.

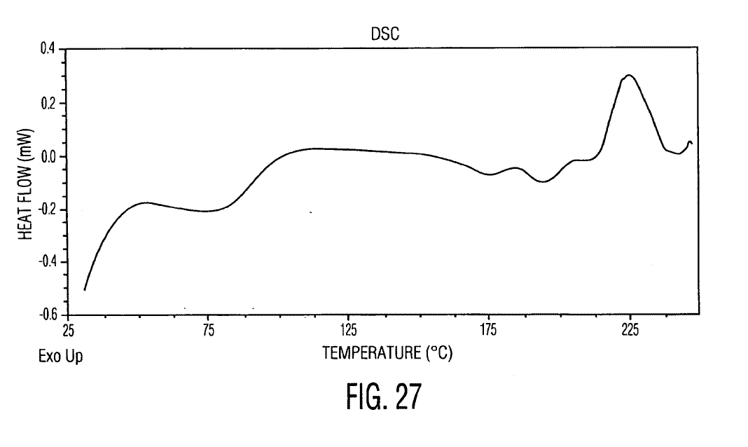

Docetaxel crystalline Form IX is further characterized by its DSC curve, which is substantially in accordance with FIG. 27, having an endothermic peak at about 173° C.

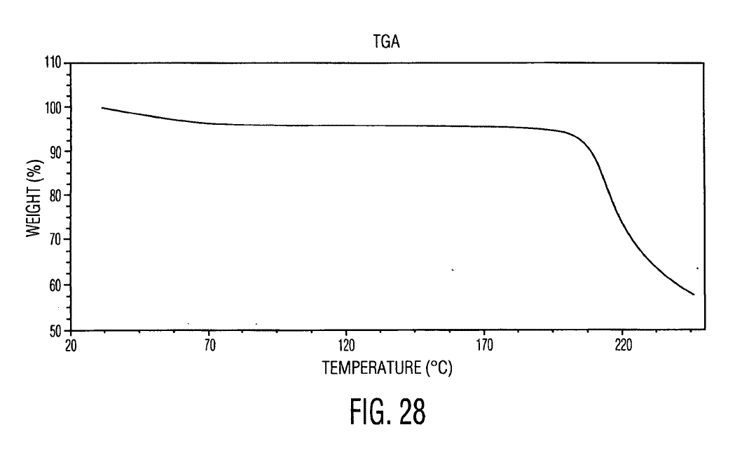

Docetaxel crystalline Form IX has a characteristic TGA curve substantially in accordance with FIG. 28, having a weight loss of about 4% w/w.

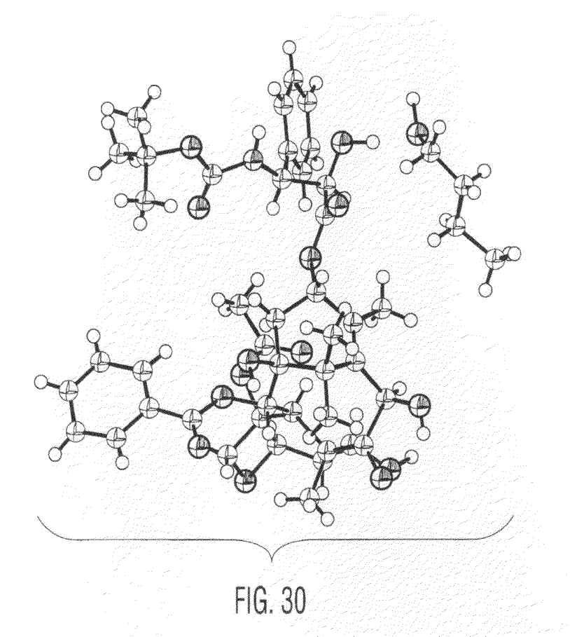

Docetaxel crystalline Form IX of the present invention is further characterized by its single crystal X-ray diffraction data (ORTEP) substantially in accordance with FIG. 30 and has the following characteristics.

Unit Cell Parameters

| | |
|---|---|
| a | 12.958(4) |
| b | 8.823(3) |
| c | 20.666(7) |
| alpha | 90 |
| beta | 98.764(4) |
| gamma | 90 |
| volume | 2335(1) |

Space group information

| | |
|---|---|
| Symbol | P21 |
| Number | 4 |
| centricity | Acentric |
| Z value | 2 |
| Formula weight | 882.01 |
| calculated density | 1.254 |
| mu (cm⁻¹) | 0.929 |
| crystal system | Monoclinic |
| laue group | 2/m |
| lattice type | P |

The present invention provides a process for making docetaxel crystalline Forms VI, Forms VII, Forms VIII and Form IX comprising:

a) providing a solution of docetaxel in a suitable organic solvent;

b) removing the solvent from the solution of step a) for formation of crystals; and

c) recovering the solid desired crystalline polymorph of docetaxel.

Step a) involves providing a solution of docetaxel in an organic solvent under suitable conditions;

The solution of docetaxel can be obtained by dissolving the compound in an organic solvent. Any form of docetaxel is acceptable for forming the solution, such as any crystalline or amorphous form of docetaxel.

Organic solvents, which can be used in the providing solution of docetaxel include but are not limited to: dimethyl sulfoxide (DMSO), acetonitrile, N,N-dimethylfomiamide (DMF), n-butanol and the like.

In an embodiment docetaxel crystalline Form VI (a DMSO solvate) is obtained when DMSO is used as solvent.

In another embodiment docetaxel crystalline Form VII (a acetonitrile solvate) is obtained when acetonitrile is used as solvent.

In a further embodiment docetaxel crystalline Form VIII (a DMF solvate) is obtained when DMF is used as the solvent.

In yet another embodiment docetaxel crystalline Form IX (an n-butanol solvate) is obtained when n-butanol is used as the solvent.

The concentration of docetaxel in the solution is not critical as long as sufficient solvent is employed to ensure total dissolution to provide a homogenous solution. The quantity of solvent used for the dissolution of the docetaxel can range from about 1 to about 25 times the weight of docetaxel taken.

The temperature for dissolution of docetaxel can range from about 0° C. to about 100° C., or the reflux temperature of the solvent used.

The solution obtained can optionally be filtered, such as by passing through filter paper, filter cloth, glass fiber, or other membrane material, or a bed of a clarifying agent such as celite, to provide the desired homogenous solution.

Step b) involves removing the solvent from the solution of step a) under suitable conditions for formation of crystals.

Removal of solvent can be carried out suitably using evaporation, atmospheric distillation or distillation under vacuum with stirring or without stirring of the solution.

Evaporation of the solvent can be conducted at temperatures from about 0° C. to about 150° C. Any temperature can be used as long as concentration occurs without an increase in impurity levels.

The time for evaporation of the solvent can be in the range of about 1 hour to about 48 hours, or longer, in the presence or absence of vacuum and in the presence or absence of an inert atmosphere such as nitrogen, argon, helium, etc.

Step c) involves recovering a solid from step b), which is the desired polymorphic form of docetaxel of Formula I.

The crystalline state of a compound can be unambiguously described by several crystallographic parameters: unit cell dimensions, space group, and atomic position of all atoms in the compound relative to the origin of its unit cell. These parameters are experimentally determined by single crystal X-ray analysis.

The results of a single crystal X-ray analysis are limited to, as the name implies, one crystal placed in the x-ray beam. Crystallographic data on a large group of crystals provides X-ray powder diffraction information. If the powder consists of a pure crystalline compound, a simple powder diagram is obtained. To compare the results of a single crystal analysis and a powder X-ray analysis, a simple calculation can be done converting the single crystal analysis and powder X-ray diagram. This conversion is possible because the single crystal experiment routinely determines the unit cell dimensions, space group, and atomic positions. These parameters provide a basis to calculate a perfect powder pattern. Comparing this calculated powder pattern and the powder pattern experimentally obtained from a large collection of crystals will confirm if the results of the two techniques are the same. This has been done for docetaxel solvated single crystals having Form VIII and Form IX.

The unit cell dimension is defined by three parameters: length of the sides of the cell, relative angles of sides to each other and the volume of the cell. The lengths of the sides of the unit cell are defined by a, b and c. The relative angles of the cell sides are defined by α, β, and γ. The volume of the cell is defined as V.

In an embodiment, the present invention provides a process for preparing an amorphous form of docetaxel, comprising precipitating amorphous docetaxel from a solution of docetaxel in THF with a hydrocarbon anti-solvent, and recovering precipitated amorphous solid.

The docetaxel solution can be prepared by the dissolution of docetaxel in THF. The concentration of docetaxel in the solution is not critical as long as sufficient THF is employed to ensure total dissolution. The amount of THF employed is usually kept small, to avoid excessive product losses during crystallization and isolation.

The quantity of THF used for the preparation of amorphous docetaxel is frequently about 1 to about 12 times the weight of docetaxel. The solution can be prepared at a temperatures ranging from about 0° C. to about 100° C. Depending on the quantity of solvent taken, a quantity of docetaxel may dissolve at ambient temperatures, or the solution may need to be heated to elevated temperatures such as about 25° C. to 100° C.

Amorphous docetaxel can be obtained by the combination of docetaxel solution with an anti-solvent.

Suitable anti-solvents which can be used in the preparation of amorphous docetaxel include but are not limited to: straight chain or branched or cyclic alkanes consisting 4 to about 10 carbon atoms such as n-hexane, n-heptane, cyclohexane, cycloheptane and the like; aromatic hydrocarbons such as benzene, toluene, xylene and the like; and mixtures thereof.

The amorphous form of docetaxel obtained from the present invention may be recovered by any method, such as decanting the solvent or by filtration or by evaporation of solvent.

The present invention also provides another process for preparing an amorphous form of docetaxel comprising removing solvent from a solution of docetaxel in an alcohol.

A docetaxel solution can be prepared by the dissolution of docetaxel in an alcohol such as ethanol, methanol, n-butanol and the like, or any combination thereof.

The solvent can be removed by any methods such as distillation, evaporation under vacuum, spray drying, ATFD, lyophilisation, flash evaporation and the like.

Suitable temperatures for the formation of amorphous docetaxel are from about 25 to about 70° C., or from about 35 to about 50° C.

A wet cake obtained after solvent removal may optionally be further dried. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any time periods necessary for obtaining a desired purity, such as from about 1 to 25 hours, or longer.

The starting material, which can be used for the preparation of polymorphic forms of the present invention, can be crude or pure docetaxel obtained by any method known in the art. The starting material for any process can be of any polymorphic form, such as crystalline forms of docetaxel, amorphous docetaxel, or mixtures of amorphous and crystalline forms of docetaxel in any proportions, obtained by any method.

The recovery can be carried out using techniques such as filtering, decanting, centrifuging and the like, or by filtering under an inert atmosphere using gases such as for example nitrogen and the like.

In another aspect, the present invention also provides a process for the preparation of docetaxel comprising:

a) reacting the compound 4-acetoxy-2α-benzoyloxy-5β,20-epoxy-1-hydroxy-9-oxo-7β,10β-bis(2,2,2-trichloroethoxycarbonyloxy)-tax-11-en-13-α-yl(4S,5R)-3-t-(butoxycarbonyl)-2,2-dimethyl-4-phenyl-5-oxazolidinecarboxylate (DCT-II) of Formula V with zinc and acetic acid to form the compound 4-acetoxy-2α-benzoyloxy-5β,20-epoxy-1β,7β,10β-trihydroxy-9-oxo-tax-11-en-13-α-yl-(4S,5R)-3-t-(butoxycarbonyl)-2,2-dimethyl-4-phenyl-5-oxazolidinecarboxylate (DCT-III) of Formula VI;

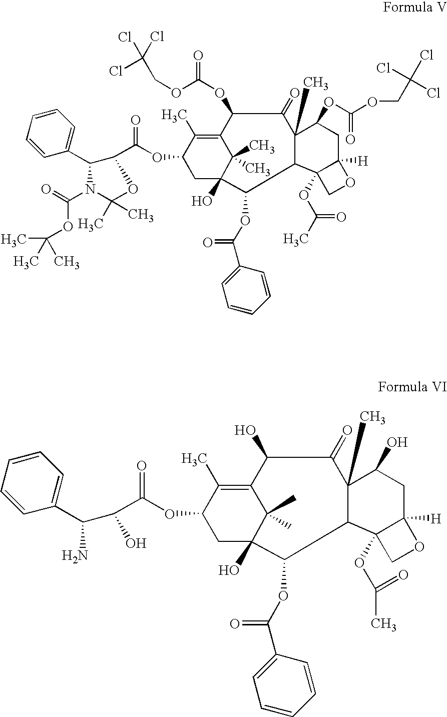

b) reacting the compound DCT-III of Formula VI with an acid to form the compound 4-acetoxy-2α-benzoyloxy-5β,20-epoxy-1β,7β,10β-trihydroxy-9-oxo-tax-11-en-13-α-yl(2R, 3S)-3-amino-2-hydroxy-3-phenyl-propionate (DCT-IV) of Formula VIA; and

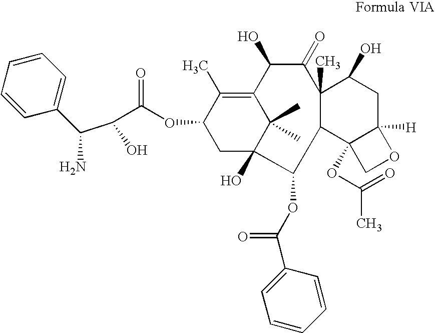

c) reacting the compound DCT-IV of Formula VIA with di-t-butyl dicarbonate to form docetaxel of Formula I.

Step a) involves reacting DCT-II of Formula V with zinc and acetic acid to form DCT-III of Formula VI.

The amount of zinc used in step a) can range from about 1 to about 10 molar equivalents, or about 8 molar equivalents, per molar equivalent of DCT-II of Formula V. The zinc can be used in any form, such as a powder, turnings, granules, etc.

The concentration of acetic acid used step a) is from about 95 to about 100%. The quantity of acetic acid can range from about 1 to about 15 L per kg of DCT-II of Formula V.

After completion of the reaction, the reaction mixture can be filtered to remove zinc and then the solid is isolated by combining the solution with an anti-solvent such as water.

Optionally, the obtained solid can be dissolved in a suitable solvent and then the solid is re-precipitated with an anti-solvent to get a desired purity.

Solvents that can be used in the dissolution include without limitation thereto ethyl acetate, isobutyl acetate, n-butyl acetate, n-propyl acetate, isopropyl acetate and the like. Anti-solvents that can be used in the precipitation include but are not limited to straight or branched alkanes or cycloalkanes consisting with C₄ to about C₁₀ such as n-pentane, n-hexane, n-heptane, cyclohexane and the like, or aromatic hydrocarbons such as benzene, toluene, xylene and the like.

DCT-III of Formula VI obtained by the above process of the present invention has a purity of not less than about 88%, or about 90%, by high performance liquid chromatography (“HPLC”).

Step b) involves reacting DCT-III of Formula VI with an acid to form DCT-IV of Formula VIA.

Suitable acids, which can be used in step b), include but are not limited to formic acid, acetic acid, triflouroacetic acid, and the like.

The quantity of acid used for in step b) can range from about 1 to about 25 L per kg of DCT-III of Formula VI.

Suitable temperatures for conducting the reaction range from about 10 to about 50° C., or about 25 to 30° C.

After completion of the reaction, the reaction mixture is concentrated and the product is extracted into a suitable solvent, and then pH is adjusted with a base in a suitable solvent.

Concentration is carried out at about 35 to about 65° C., or about 40 to about 45° C., for the formation of residue.

Water is added to the residue and the obtained solution is washed with a suitable organic solvent such as methyl isobutyl ketone (MIBK), ethyl acetate, isobutyl acetate, n-butyl acetate, n-propyl acetate, isopropyl acetate, dichloromethane, chloroform, and the like.

Aqueous layer pH is adjusted with a suitable base to a value between 7.5 and about 10. Suitable bases used in the pH adjustment include but are not limited to inorganic bases such as sodium hydroxide, sodium carbonate, sodium bicarbonate, sodium acetate, potassium hydroxide, potassium carbonate, potassium bicarbonate and the like; organic bases such as triethyl amine, diisopropyl amine, and the like.

The compound of DCT-IV of Formula VIA is optionally not isolated from the reaction mixture. It can be progressed directly for conversion into docetaxel in the next step.

Step c) involves reacting DCT-IV of Formula VIA with di-t-butyl dicarbonate to form docetaxel of Formula I.

The quantity of di-t-butyl dicarbonate used in the formation of docetaxel is from about 1 to about 4 molar equivalents, or about 3 molar equivalents, per molar equivalent of DCT-III of Formula VI (if DCT-IV was not isolated), or per molar equivalent of DCT-IV of Formula VIA.

Suitable solvents that can be used in the formation of docetaxel include but are not limited to water, ethyl acetate, isobutyl acetate, n-butyl acetate, n-propyl acetate, isopropyl acetate, dichloromethane, chloroform, and the like, and mixtures thereof.

Step c) can be carried out at temperatures from about 10 to about 65° C., or from about 25 to about 35° C.

After completion of the reaction, the organic layer is separated and concentrated to a suitable volume. Concentration may be carried out suitably using techniques such as evaporation, atmospheric distillation, distillation under vacuum, or agitated thin film drying (“ATFD”). Concentration typically will be terminated when the docetaxel concentration reaches about 0.1 g/ml to about 0.5 g/ml, or about 0.3 g/ml

The solid can be isolated from the concentrated reaction solution by combining with an anti-solvent. Useful anti solvents include but are not limited to not limited to straight or branched aliphatic alkanes or cycloalkanes of C₄ to about C₁₀ such as n-hexane, n-heptane, cyclohexane and the like, or aromatic hydrocarbons such as benzene, toluene, xylene and the like.

Docetaxel obtained by the present process typically has a purity not less than about 75% or about 80% by HPLC.

The obtained docetaxel from the present invention can be purified using column chromatography with silica gel, eluting using an eluent, and then again purifying using recrystallization from suitable solvents.

The silica gel, which can be used for the purification, can have a particle size range such as for example 230-400 mesh, 100-200 mesh, 60-100 mesh, or 500-750 mesh.

Suitable eluents include but are not limited to ethyl acetate, isobutyl acetate, n-butyl acetate, n-propyl acetate, isopropyl acetate, n-heptane, n-hexane, cyclohexane and combinations thereof.

The solid can be recovered from pure eluent fractions by techniques such as evaporation, atmospheric distillation, distillation under vacuum, or agitated thin film drying (“ATFD”) and the like.

Docetaxel obtained by the above column chromatography process of the present invention typically has a purity not less than about 90%, or about 94%, by HPLC.

The obtained docetaxel from column chromatography can be further purified by combining a solution of docetaxel in a ketone with an anti-solvent.

Docetaxel solutions can be prepared by the dissolution of docetaxel in a ketone solvent. Useful ketone solvents include, without limitation, acetone, methyl isobutyl ketone, methyl ethyl ketone, and the like.

The concentration of docetaxel in the solution is not critical, but the quantity of solvent employed is usually kept to a minimum so as to avoid excessive product losses during the crystallization of solid. The concentration of docetaxel in the solution may generally range from about 0.01 to about 0.25 g/ml in the solvent.

The solution can be prepared at temperatures ranging from about 25° C. to 100° C. Depending on the quantity of solvent taken, docetaxel may dissolve at 25 to 35° C., or the solution may need to be heated to elevated temperatures of about 40° C. to 55° C.

A decolorizing carbon treatment can be optionally given either at the dissolution temperatures or after cooling the solution to lower temperatures.

The solid can be crystallized from reaction solution by combining with an anti-solvent. Useful anti-solvents include ethers such as diethyl ether, diisopropyl ether, tetrahydrofuran (THF), 1,4-dioxane, dimethoxyethane, methyl tertiary-butyl ether and the like.

Suitable temperatures for solid crystallization can range from about 20 to about 80° C., or about 25 to about 35° C.

The above recrystallization process can be repeated one or more times to get a purity typically greater than or equal to about 99%, or about 99.5%, by weight as determined using HPLC.

The obtained docetaxel is optionally slurried in a suitable solvent to get a desired level of residual solvents content, frequently expressed in ppm as determined by gas chromatography (“GC”). Suitable solvents include n-heptane, n-hexane, cyclohexane and the like.

Suitable temperatures range from 20 to about 40° C., or from about 25 to about 35° C.

The slurry can be maintained for periods of about 20 minutes to about 4 hours, or longer.

The wet solid may optionally be further dried. The drying can be carried out at temperatures of about 35° C. to about 70° C. for any time periods necessary for obtaining a desired purity, such as from about 1 to 25 hours, or longer.

The docetaxel polymorphs of present invention are stable and are well suited for use in preparing pharmaceutical formulations. The pharmaceutical formulations according to the present invention include but are not limited to solid oral dosage forms such as tablets, capsules, powders and so on; liquid oral dosage forms such as solutions, dispersions, suspensions, emulsions and so on; parenteral dosage forms (including intramuscular, subcutaneous, intravenous) such as injectable dosages by solution or suspension or dispersions or sterile powders for reconstitution; transdermally delivery systems; targeted delivery systems etc.

For solid oral dosage forms the composition comprises excipients which include but are not limited to diluents, disintegrants, binders, lubricants, flavoring agents, coloring agents and so on. For liquid oral dosage forms the composition include but not limited to pharmaceutically acceptable aqueous or non aqueous vehicles etc., flavoring agents, preservatives, solubilizers, emulsifiers and soon.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical formulations may be adapted for topical administration included but not limited to ointments, creams, suspensions, lotions, powders, solutions, pastes, gels, sprays, aerosols or oils.

The pharmaceutical formulations can further be packed in vials or ampoules made of glass, containers and lids of high-density polyethylene (HDPE), low-density polyethylene (LDPE) and or polypropylene and/or glass, and blisters or strips composed of aluminium or high-density polypropylene. These lists are not intended to be exhaustive, as other materials and packaging types are also useful.

The process for the preparation of docetaxel and processes for the preparation of polymorphs of docetaxel of the present invention are simple, give a product having a high melting point and more stability, and are cost effective, reproducible, robust and industrially scalable.

Certain specific aspects and embodiments of the present invention will be explained in more detail with reference to the following examples, which are provided by way of illustration only and should not be construed as limiting the scope of the invention in any manner.

### Example 1

### Preparation of Crystalling Form I of Docetaxel

126 g of docetaxel was dissolved in 1.35 L of acetone and precipitated by adding 8.1 L of petroleum ether. The mixture was stirred for 90 minutes at 27° C. and the solid suspension was filtered. The solid was dried for 3 hours at 30° C. under a vacuum of 600 mm Hg to afford 121 g of title compound.

Purity: 99.6% by HPLC.

Water content: 1.98% w/w by the KF method.

### Example 2

### Preparation of Crystalling Form II of Docetaxel

300 g of docetaxel was dissolved in 3 L of ethanol at 27° C. and filtered. The filtrate was concentrated to dryness at 50° C. under a vacuum of 680 mm Hg for 1 hour. The obtained solid was dried at 35° C. under vacuum of 650 mm Hg for 4 hours. The solid was suspended in 3 L of acetonitrile and heated to 40° C. The obtained solution immediately charged into 14.5 L of preheated water at 40° C. The mixture was stirred for 4 hours at 45° C. and then the solid suspension was filtered. The obtained solid was washed with 2 L of water. The wet solid was charged into 8.7 L of water and stirred for 1 hour at 25° C. The suspension was filtered and washed with 2 L of water. The above water slurrying procedure was repeated 3 times and the resultant solid was dried at 35° C. for 2 hours under a vacuum of 680 mm Hg. The obtained solid was exposed to 80±2° C. relative humidity (RH) and a temperature 25±2° C. for about 36 hours to afford 273 g of title compound.

Purity: 99.67% by HPLC.

Water content: 6.7% w/w by KF.

### Example 3

### Preparation of Crystalling Form III of Docetaxel

1 g of docetaxel (Form II) and 5 ml of isopropyl alcohol were charged into a clean and dry round bottom flask and stirred for 1 hour. The obtained solid suspension was filtered and dried under a vacuum of 680 mm Hg at 50° C. for 24 hours to afford 0.65 g of the title compound.

Purity: 99.69% by HPLC.

Water content: 6.3% w/w by KF.

### Example 4

### Preparation of Crystalling Form IV of Docetaxel

1 g of docetaxel (Form II) was charged into a clean and dry round bottom flask. 2 ml of DMF was charged into the flask and the mixture was stirred for 1 hour. The resultant clear solution was precipitated by adding 20 ml of water. The obtained reaction suspension was filtered and dried under a vacuum of 680 mm Hg for 24 hours at 50° C. to afford 0.85 g of title compound.

Purity: 98.47% by HPLC.

Water content: 1.0% w/w by KF.

### Example 5

### Preparation of Crystalling Form V of Docetaxel

70 ml of toluene was charged into a clean and dry round bottom flask at 27° C. A solution of docetaxel (0.6 g of Form II dissolved in 2.5 ml of THF) was added to the toluene over a period of 10 minutes and stirred for 1 hour at 27° C. The obtained precipitate was filtered and dried under a vacuum of 680 mm Hg for 30 hours at 50° C. to afford 0.514 g of title compound.

Purity: 99.6% by HPLC.

Water content: 4.28% w/w by KF.

### Example 6

### Preparation of Amorphous Docetaxel by Precipitation

70 ml of n-hexane was charged into a clean and dry round bottom flask at 27° C. Docetaxel solution, prepared by the dissolution of 0.6 g of docetaxel in 2.5 ml of THF, was added to the n-hexane over a period of 10 minutes and stirred for 1 hour for precipitation. The obtained precipitate was filtered and dried under a vacuum of 680 mm Hg at 50° C. for 30 hours to afford 0.532 g of title compound.

Purity: 99.63% by HPLC.

### Example 7

### Preparation of Amorphous Docetaxel Using Distillation

1 g of docetaxel and 10 ml of ethanol were charged into a clean and dry round bottom flask at 27° C. The resultant solution was heated to 50° C. and the clear solution was concentrated to dryness under a vacuum of 650 mm Hg. The above procedure was repeated 3 times and then a vacuum of 600 mm Hg was applied for 30 minutes to get a dry solid. The obtained solid was dried under a vacuum of 600 mm Hg at 50° C. for 24 hours to afford 0.82 g of title compound.

Purity: 99.45% by HPLC.

### Example 8

### Preparation of Crystalling Form VI of Docetaxel

5 ml of DMSO and 1 g of docetaxel were charged into a round bottom flask with simultaneous stirring at 27° C. and stirred for 10 minutes. The obtained solution was allowed to stand without disturbance at 27° C. for 7 days to evaporate DMSO from the solution. The obtained crystals were filtered and suction dried for 2 days under a vacuum of 650 mm Hg at 30° C. to afford the title compound.

### Example 9

### Preparation of Crystalling Form VII of Docetaxel

5 ml of acetonitrile and 1 g of docetaxel were charged into a round bottom flask with stirring at 27° C. The solution was stirred for 10 minutes. The obtained solution was allowed to stand without disturbance at 27° C. for 6 days to evaporate acetonitrile from the solution. The obtained crystals were filtered under a vacuum of 680 mm Hg and suction dried for 3 days under a vacuum of 600-680 mm Hg at 25-30° C. to afford crystalline Form VII.

### Example 10

### Preparation of Crystalling Form VIII of Docetaxel

5 ml of DMF and 1 g of docetaxel were charged into a round bottom flask with stirring at 27° C. The solution was stirred for 10 minutes. The solution was allowed to stand without disturbance at 27° C. for 5 days to evaporate DMF from the solution, then the crystals were filtered and suction dried for 50 hours at 27° C. under a vacuum of 650 mm Hg to afford the title compound.

FIG. 29 is an ORTEP depiction of the product docetaxel and DMF solvate (1:1) drawn at a 50% probability level for non-hydrogen atoms. Hydrogen atoms are omitted for clarity.

### Example 11

### Preparation of Crystalling Form IX of Docetaxel

5 ml of n-butanol and 1 g of docetaxel were charged into a round bottom flask with stirring at 27° C. and stirred for 10 minutes. The obtained solution was allowed to stand without disturbance at 27° C. for 6 days to evaporate n-butanol from the solution, then the crystals were filtered and suction dried for 50 hours at 27° C. under a vacuum of 650 mm Hg to afford the title compound.

FIG. 30 is an ORTEP depiction of the product docetaxel and n-butanol solvate (1:1) drawn at a 50% probability level for non-hydrogen atoms.

### Example 12

### Preparation of 4-Acetoxy-2α-Benzoyloxy-5β,20-Epoxy-1β,13α-Dihydroxy-9-Oxo-7β,10β-Bis(2,2,2-Trichloroethoxycarbonyloxy)-11-Taxen of Formula III

125 ml of pyridine and 25 g of 10-DAB III (10-deacetyl baccatine III) were charged into clean and dry round bottom flask. Stirred the solution for 15 minutes under a nitrogen atmosphere and a solution of Troc-Cl (2,2,2-trichloroethoxycarbonyl chloride), prepared by the dilution of 22.6 ml of Troc-Cl with 250 ml of dichloromethane, was added slowly over a period of 1 hour under a nitrogen atmosphere. Stirred the mixture for 5 minutes and checked reaction completion using thin layer chromatography (TLC) to confirm the consumption of the starting material. 500 ml of demineralized water was charged to the reaction mixture and stirred for 5 minutes. Separated the organic layer from the mixture and then washed the organic layer with 1750 ml of 10% HCl solution three times. Organic layer was washed with 500 ml of saturated NaHCO₃ solution followed by 500 ml of demineralized water and then the obtained organic layer was concentrated to a volume of 97.5 ml at 45° C. under a vacuum of 650 mm Hg. 250 ml of toluene was charged to the concentrate and it was again concentrated to a volume of 87.5 ml. The concentrate was stirred for 2 hours at 0° C. and then the solid was separated by filtration to afford 36 g (yield: 87.8%) of title compound. Purity: 96.76% by HPLC.

### Example 13

### Preparation of DCT-II of Formula V

1500 ml of dichloromethane and 80.96 g of (4S,5R)-3-tertiary-(butoxycarbonyl)-2,2-dimethyl-4-phenyl-5-oxazolidinecarboxylate was taken into a round bottom flask. 150 g of 4-acetoxy-2α-benzoyloxy-5β,20-epoxy-1β,13α-dihydroxy-9-oxo-7β,10β-bis(2,2,2-trichloroethoxycarbonyloxy)-11-taxen (from Example 12) was charged under a nitrogen atmosphere to the solution. 20.5 g of dimethylaminopyridine (DMAP) and 103.9 g of dicylohexyldicarbamide (DCC) were charged to the solution and stirred for 1 hour. The reaction mixture was filtered and washed with 300 ml of dichloromethane. The obtained filtrate was washed with 750 ml of saturated NaHCO₃ solution and again washed with 1500 ml of demineralized water. The obtained organic layer was concentrated to a volume of 450 ml at 45° C. under vacuum of 580 mm Hg. Filtered the organic layer to remove by-product dicyclohexylurea and the solid was washed with 300 ml of dichloromethane. 3000 ml of n-heptane was charged into another round bottom flask at 25° C. The obtained filtrate was added slowly to the n-heptane over a period of 40 minutes and then stirred for 1 hour. The suspension was filtered and the solid was washed with 300 nil of n-heptane. The solid was dried at 50° C. for 4 hours under a vacuum of 680 mm Hg to afford 182.5 g (yield: 93.7%) of title compound.

Purity: 98.84% by HPLC.

### Example 14

### Preparation of DCT-III of Formula VI

1250 ml of acetic acid and 125 g of DCT-II were charged into a clean and dry round bottom flask with stirring. Stirred the solution for 10 minutes at 26° C. and then 1250 ml of methanol was charged to the solution. 54.3 g of zinc dust was charged to the solution and then heated to 55° C. Stirred the mixture for 20 minutes at 58° C. Prepared a celite bed with 65 g of celite in 250 ml of methanol. The reaction mixture was filtered through the celite bed and the bed washed with 250 ml of methanol. The filtrate was slowly added to 8.75 L of water over a period of 5 minutes and then the suspension was stirred for 1 hour at 27° C. The suspension was filtered and the solid was washed with 250 ml of water. The solid was dissolved in 1250 ml of ethyl acetate and then the ethyl acetate layer was washed with 125 ml of water. Organic layer was filtered and then the filtrate was concentrated to a volume of 500 ml at 50° C. 2.5 L of n-heptane was added slowly over a period of 15 minutes at 27° C. and then the suspension was stirred for 1 hour. The suspension was filtered and the solid washed with 250 ml of n-heptane and dried at 50° C. for 4 hours to afford 76.6 g (yield: 86.7%) of title compound. Purity: 90.54% by HPLC.

### Example 15

### Preparation of Docetaxel of Formula I

500 ml of formic acid was taken into a round bottom flask and cooled to 22° C. 50 g of DCT-III was added and stirred for 1.5 hours. The solution was concentrated at 42° C. under a vacuum of 580 mm Hg to obtain a residue. 500 ml of MIBK and 500 ml of water were charged to the residue and then stirred for 10 minutes. The aqueous layer was separated and washed with 500 ml of MIBK. Again, the aqueous layer was washed with ethyl acetate (2×500 ml) and then pH was adjusted to 8.4 by adding 54 g of NaHCO₃. 20 ml of di-t-butyl dicarbonate was added to the aqueous layer and stirred for 30 minutes. 500 ml of ethyl acetate and 20 ml di-t-butyl dicarbonate were charged to the mixture and the mixture was stirred for 30 minutes. The organic layer was separated and concentrated to a volume of 100 ml. A solid was precipitated by adding 500 ml of n-heptane and stirring for 1 hour. The suspension was filtered and the solid dried for 30 minutes at 27° C. to afford 34 g of title compound.

Purity: 80.43% by HPLC.

### Example 16

### Purification of Docetaxel Using Column Chromatography

A column was packed with 625 g of silica gel in 2 L of 20% of ethyl acetate in n-heptane. 25 g of docetaxel, prepared according to Example 15, was dissolved in 50 ml of ethyl acetate and charged to the column. The column was eluted with a mixture of ethyl acetate and n-heptane: 2L of 20% ethyl acetate and 20 L of 50% ethyl acetate. After elution of 11 L, a purified fraction of 8.5 L was collected. The purified fraction was concentrated completely at 47° C. under a vacuum of 680 mm Hg to afford 16.2 g of title compound.

Purity: 94.47% by HPLC.

### Example 17

### Purification of Docetaxel

1370 ml of acetone and 137 g of docetaxel were charged into a clean and dry round bottom flask. The mixture was heated to 45° C. and then stirred for 30 minutes. The solution was filtered and the filtrate was cooled to 27° C. 4110 ml of diisopropyl ether and 100 mg of pure docetaxel were charged to the solution. The suspension was stirred for 1.5 hours and then filtered. The solid was washed with 275 ml of diisopropyl ether and dried at 60° C. for 4 days under a vacuum of 680 mm Hg to afford 92 g of the title compound.

Purity: 99.38% by HPLC.

### Example 18

### Preparation of Crystalling Form I of Docetaxel

140 ml of acetone and 7 g of docetaxel were charged into a round bottom flask. The solution was stirred for 10 minutes and filtered. The filtrate and 420 ml of diisopropyl ether were charged into a round bottom flask. 50 mg of docetaxel, obtained according to Example 1, was charged to above flask and then stirred for 90 minutes. The suspension was filtered and the solid washed with 35 ml of diisopropyl ether. The solid was dried for 2 days at 27° C. under a vacuum of 680 mm Hg to afford 5.4 g of the title compound.

45 ml of n-heptane and 4.5 g of docetaxel, obtained above, were charged into a round bottom flask. The suspension was stirred for 15 minutes and then filtered. The solid was washed with 4.5 ml of n-heptane. The above process was repeated two more times and then the solid was dried at 50° C. for 48 hours under a vacuum of 680 mm Hg to afford 4.25 g of title compound.

Purity: 99.82% by HPLC.

Diisopropyl ether content: 96 ppm.

1. A process for preparing docetaxel, comprising reacting a compound having a formula: 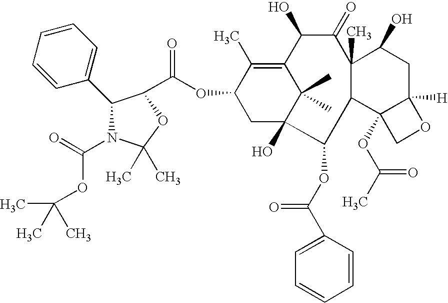
with an acid, to form a compound having a formula:

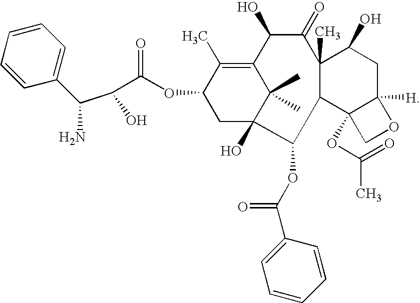

2. The process of claim 1, further comprising reacting a compound having a formula: 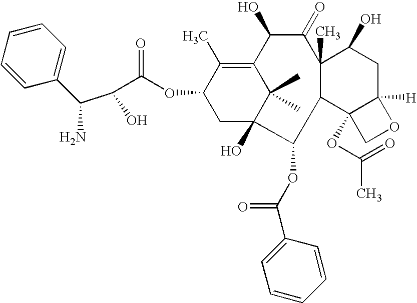
with di-t-butyl dicarbonate to form docetaxel.

3. The process of claim 1, wherein a compound having a formula: 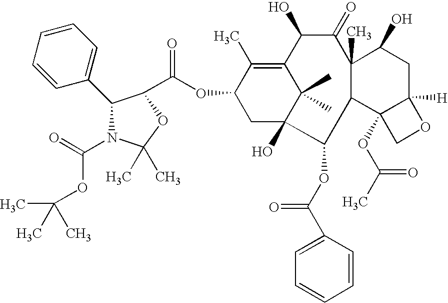
is prepared by reacting a compound having a formula 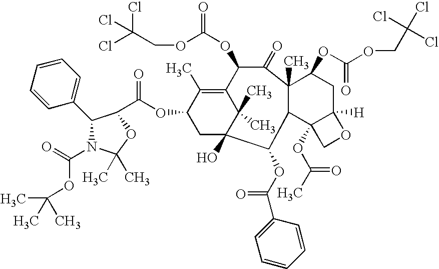
with zinc and acetic acid.

4. The process of claim 2, wherein a compound having a formula: 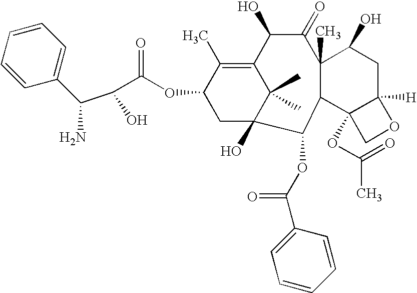
is not isolated before further reaction.

5. A process for preparing crystalline Form I of docetaxel, comprising combining a solution of docetaxel in a ketone with an anti-solvent.

6. The process of claim 5, wherein an antisolvent comprises an ether or a hydrocarbon.

7. A process for preparing crystalline Form II of docetaxel, comprising combining a solution of docetaxel in acetonitrile with water.

8. A process for preparing crystalline Form III of docetaxel, comprising slurrying solid docetaxel in isopropyl alcohol.

9. A process for preparing crystalline Form IV of docetaxel, comprising combining a solution of docetaxel in N,N-dimethylformamide with water.

10. A process for preparing crystalline Form V of docetaxel, comprising combining a solution of docetaxel in tetrahydrofuran with toluene.

11. A process for preparing a crystalline polymorphic form of docetaxel, comprising:
a) providing a solution of docetaxel in an organic solvent;
b) removing solvent from the solution of a) for formation of crystals; and
c) recovering a solid crystalline polymorph of docetaxel.

12. The process of claim 11, wherein a solvent comprises dimethyl sulfoxide and crystalline Form VI of docetaxel is obtained.

13. The process of claim 11, wherein a solvent comprises acetonitrile and crystalline Form VII of docetaxel is obtained.

14. The process of claim 11, wherein a solvent comprises N,N-dimethylformamide and crystalline Form VIII of docetaxel is obtained.

15. The process of claim 11, wherein a solvent comprises n-butanol and crystalline Form IX of docetaxel is obtained.

16. Crystalline Form I of docetaxel.

17. Crystalline Form II of docetaxel.

18. Crystalline Form III of docetaxel.

19. Crystalline Form IV of docetaxel.

20. Crystalline Form V of docetaxel.

21. Crystalline Form VI of docetaxel.

22. Crystalline Form VII of docetaxel.

23. Crystalline Form VIII of docetaxel.

24. Crystalline Form IX of docetaxel.

25. A process for preparing solid amorphous docetaxel, comprising combining a solution of docetaxel in tetrahydrofuran with an anti-solvent.

26. The process of claim 25, wherein an anti-solvent comprises a hydrocarbon.

27. A process for preparing solid amorphous docetaxel, comprising removing solvent from a solution of docetaxel in an alcohol.
